(19) United States
(12) Patent Application Publication
(10) Pub. No.: US 2004/0111043 A1
(43) Pub. Date: Jun. 10, 2004

(54) Bioelectric telemetering system and method for digital cable eliminator

(75) Inventors: Imre Szabo, Pecs (HU); Kalman Mathe, Pecs (HU); Andre Fenton, New York, NY (US)

Correspondence Address: THE FIRM OF KARL F ROSS, 5676 RIVERDALE AVENUE, PO BOX 900, RIVERDALE (BRONX), NY, 10471-0900, US

(73) Assignee: Bio-Signal Group Corp.

(21) Appl. No.: 10/425,023

(22) Filed: Apr. 28, 2003

Related U.S. Application Data
(63) Continuation-in-part of application No. 10/314,890, filed on Dec. 9, 2002.

(51) Int. Cl. A61B 5/04

(52) U.S. Cl. 600/544; 128/903

(57) ABSTRACT

Bioelectrical activity in a living animal or human subject is monitored by detecting the bioelectrical signals at a number of points digitizing and multiplexing the electrical signals and transmitting the multiplexed stream by digital radio transmission circuitry. The received data stream is then demultiplexed and monitored to allow the activity at the points on the animal subject to be monitored.

### CROSS REFERENCE TO RELATED APPLICATION

This application is a continuation-in-part of copending application Ser. No. 10/314,890 filed 9 Dec. 2002.

### FIELD OF THE INVENTION

Our present invention relates to a bioelectric telemetering system and method.

### BACKGROUND OF THE INVENTION

In the past for the measurement of biological signals, it has been the pattern to provide bioelectrical transducers at the site at which the biological signals are generated and to connect those transducers by cables to recording and analyzing circuits. Animal studies have been widely carried out with this form of bioelectrical signal transmission and this approach has also been applied to neurophysiological signal transmission in monitoring of the brains of laboratory animals, or animal test subjects. This approach has also been used for monitoring bioelectric signals from human subjects in the study and pre-surgical monitoring of diseases such as epilepsy.

An obvious disadvantage of this approach is that it is not able to transmit signals from free or untethered subjects. This may affect the behavior that produces the neurophysiological response of study and may complicate or even prohibit the intended study. For example, in human intracranial recordings, the tension generated by the tether to the recording electronics may cause tissue damage if the subject movements are uncontrolled, for example during a seizure.

### OBJECTS OF THE INVENTION

It is, therefore, the principal object of the present invention to provide an improved method of monitoring bioelectric events in subjects whereby this drawback is avoided.

A second object of the invention is to provide an inexpensive method for high quality recording of bioelectric signals.

A third object of the invention is to provide a method of monitoring biological signals in subjects which are not limited by cable tethers of the subject and nevertheless can ensure a high reliability and versatility of signal evaluation.

A fourth object of this invention is to provide a method to wirelessly transmit signals from conventional transducers such as those from for example a gyroscope, or accelerometer, to record signals such as acceleration, movement, tremor, body temperature, blood pressure, pulse rate, blood carbon dioxide level, skin resistance, speech, etc.

A fifth object of this invention is to provide a telemetering system for an animal subject which minimizes the physical inhibition of the animal subjects.

### SUMMARY OF THE INVENTION

These objects are attained, in accordance with a feature of this invention, by providing an audio frequency pickup for bioelectrical signals at a site on the subjects to be monitored. That signal is digitized multiplexed and arranged as a digital audio standard. The information is transmitted by radio frequency transmission to a location spaced from the subject, where the information and thereafter processing the information at least in part by recording and displaying the signal individual to the original transducer or signal source.

According to a feature of the invention, each pair of such signals is conditioned by a pair of analog amplifiers, and fed to a two-channel audio input of an analog-to-digital converter, e.g. a 16 or 24 bit ADC, which digitizes and multiplexes the two analog inputs into a single digital data stream in the commercially available stereo audio ADC's. According to a feature of the invention, each such data stream carries two signals, and can be further multiplexed with other data streams in a serializing stage. According to another feature of the invention, each step of the serializing process takes a number of data streams in standard digital audio format and combines them in such a fashion as to produce in turn a single digital data stream that maintains the same standard digital audio format. The result is a single standard digital data format which can be processed by consumer audio electronic devices that adhere to the data standard. For example, the data standard can be the I2S digital audio format.

The serialized data stream can be transmitted by wire or according to a feature of the invention it can be transmitted by a radio frequency signal.

The signal is picked up by the receiver which can deserialize the information into its constituent signals. Each fully deserialized digital channel can then be submitted to digital signal processing and storage.

To ensure the invention's general utility, the digital signal can also be converted back to an analog signal (by one or more digital-to-analog converters) (DAC) for recording by conventional analog devices.

Through the use of standard digital audio integrated circuits, standard format audio inputs can be supplied to digital radio transmitter circuits which can communicate over any required distance with the receiver. The transmitter and audio input system are miniaturized already in digital audio and microwave radio technology and thus can be easily mounted on the subject or implanted in the animal subject.

The power for driving the unit on the subject may be supplied exclusively by battery, although a DC voltage source can be used for driving the transmitter.

According to a feature of the invention, the method provides for the short distance telemetric monitoring of several subjects, for example patients in a hospital which allows each subject to be totally electrically isolated from the shock hazard of power lines (e.g. 120-240 VAC) that power the machines which receive and process the signals of interest.

According to this feature of the invention an inexpensive method is provided for electrically isolating the subject being measured from the hazard of electrical shock from power lines that supply, for example monitoring and resuscitation equipment. For example, with the invention, during surgery a subject can be electrically, and physically isolated from the devices that monitor the subject's physiological signs, providing protection to both the patient and the surgical team from the shock hazard of power lines. The cable eliminator would allow the surgical team to move more freely and remove the need to sterilize the eliminated wires.

While the invention can be used to transmit the signal from conventional transducers measuring points at the subject, the invention also comprises a method of monitoring neural activity which comprises:

(a) connecting electrodes to neural activity points of an animal subject and obtaining analog signals representing activity at the points;

(b) securing to the subject an apparatus having audio analog-to-digital converters capable of digitizing and multiplexing multiple pairs of analog inputs each into respective single digital data streams which are serialized into a single data stream, and a gigahertz radiofrequency transmitter for the data streams and connecting the audio analog-to-digital converters to the electrodes, whereby digital data streams representing digitized and multiplexed signals from the electrodes are transmitted by the apparatus;

(c) at a receiver separated from the subject receiving the digital data streams transmitted by the apparatus, deserializing and demultiplexing channels of the received digital data streams to produce digital channels corresponding to the analog inputs, and converting the digital channels to analog outputs; and

(d) recording the digital or analog outputs in a multichannel digital or analog recorder having recording channels assigned to the respective electrodes.

The apparatus can comprise:

a multiplicity of electrodes connected to neural activity points of a subject and obtaining analog signals representing activity at the points;

a digital radiofrecuency transmitter apparatus mounted on the subject and having audio analog-to-digital converters connected to the electrodes and each capable of digitizing and multiplexing two analog inputs each into respective single digital data streams, and a gigahertz radiofrequency transmitter for the data streams and connected to the audio analog-to-digital converters, whereby digital data streams representing digitized and multiplexed signals from the electrodes are transmitted by the apparatus;

a receiver separated from the animal subject receiving the digital data streams transmitted by the apparatus, and provided with a deserializer for deserializing channels of the received digital data streams to produce digital channels corresponding to the analog inputs, and digital-to-analog converters for converting the digital channels to analog outputs; and

a multichannel analog recorder connected to the receiver and having recording channels assigned to the respective electrodes.

### BRIEF DESCRIPTION OF THE DRAWING

The above and other objects, features, and advantages will become more readily apparent from the following description, reference being made to the accompanying drawing in which:

FIG. 1 is an elevational view showing an animal equipped with the transmitter portion of the device (top);

FIG. 2 is a diagram showing the circuitry attached to the skull of the animal;

FIGS. 3 and 4 are perspective views showing the circuitry provided with a battery for the transmitter portion;

FIGS. 5, 6 and 7 are graphs of pulses for explanation of the invention; and

FIG. 8 is a diagram of the overall system incorporating two embodiments of the transmitter stage.

### SPECIFIC DESCRIPTION

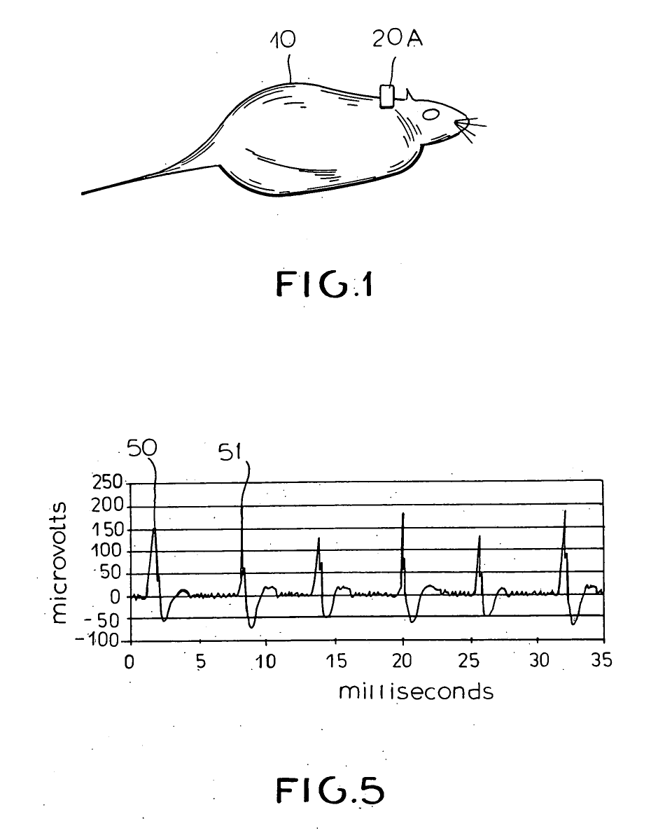

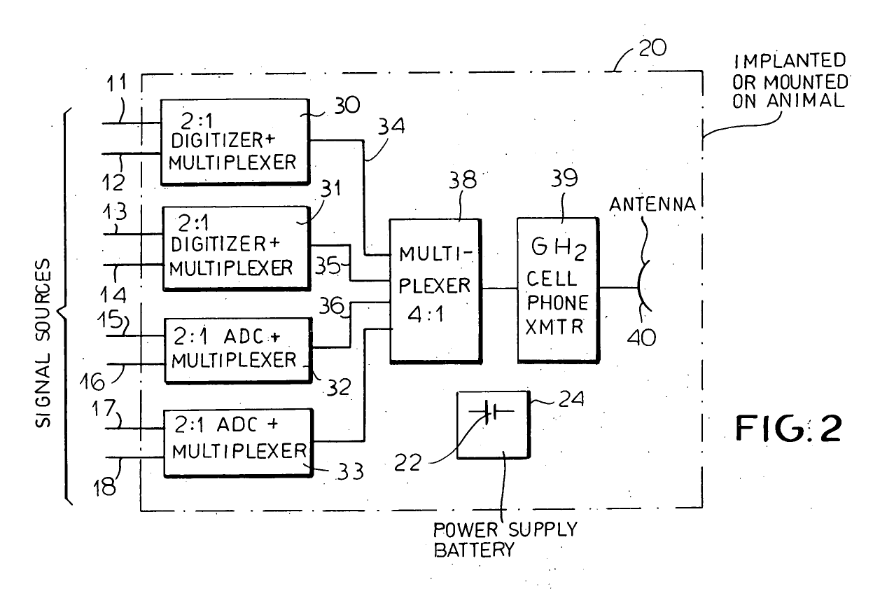

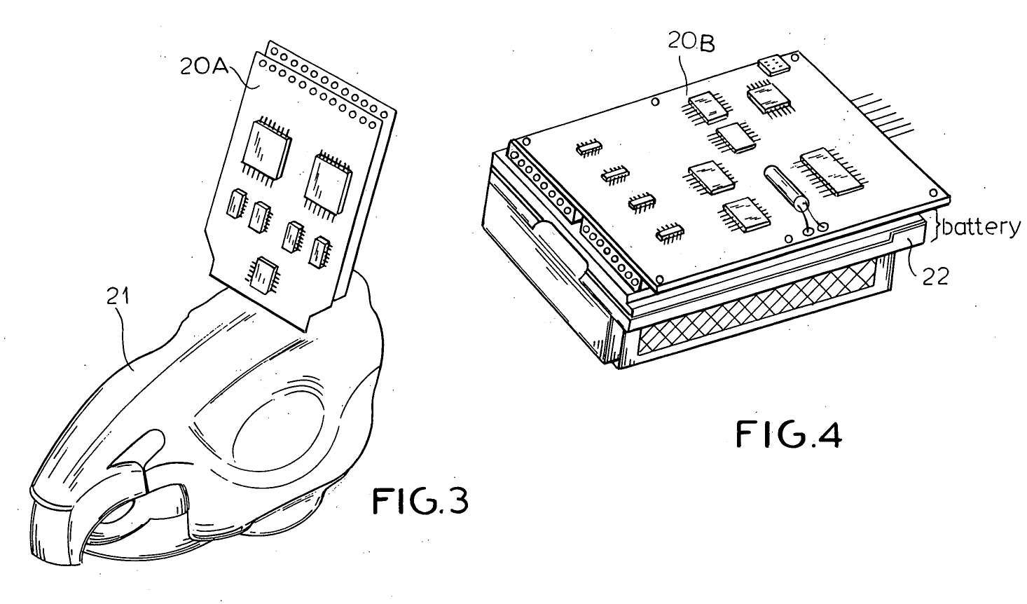

Essentially the present invention utilizes standard digital audio technology to effect telemetering of inputs from a live subject 10 (FIG. 1), here shown as a rat, for hippocampal or other studies, utilizing electrodes 11, 12, 13, 14, 15, 16, 17 and 18 (FIG. 2) which can be implanted in the brain of the animal. FIG. 3 shows the headstage form of the transmitter unit 20A affixed directly to the skull 21 of the animal and FIG. 4 shows the backpack form of the transmitter unit 20B which receives the signals via short wires from electronics affixed to the skull. That transmitter circuits 20A, 20B may have a 3.6 V battery 22 (FIG. 2).

In FIG. 2, the power supply 24 has been shown to include the battery 22.

Basically, the transmitter unit 20 (FIG. 2) can include a two-channel 24-bit delta-sigma analog to digital converter with digital 2:1 multiplexer 30, 31, 32, 33 for combining the two analog inputs 11, 12, etc. (of each pair of inputs) into a single digital data stream which is delivered via line 34, 35, 36, 37 to a 4:1 digital multiplexer called a serializer. The digital output is conveyed to a wideband frequency-modulated gigahertz oscillator and radio frequency transmitter 39 and its antenna 40.

The inputs 11 and 12 etc. may each derive from electrodes or any bioelectrical or conventional transducer capable of producing bioelectrical signals relevant to the state of the subject.

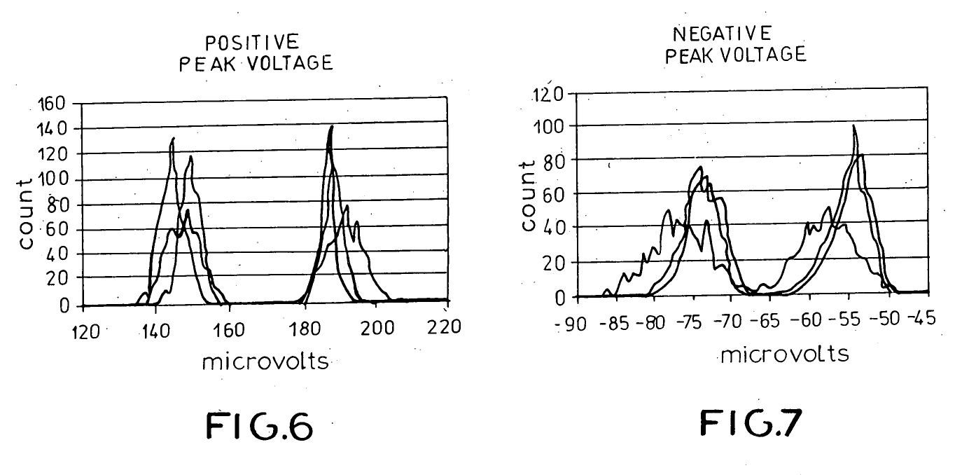

Once the signals have been conditioned by conventional analog circuits, for example by follower-configured operational amplifiers to convert input impedance field effect transistor (FET) or instrumentation amplifiers to obtain both impedance conversion and common-mode reduction, the signals which are received by the invention may have slow and fast frequency components, such as neuronal action potentials as shown at 50 and 51 in FIG. 5 in which artificially generated spikes have been plotted in microvolts along the ordinate against milliseconds along the abscissa. It is possible for an accumulation of spikes to be analyzed utilizing histograms as shown in FIGS. 6 and 7.

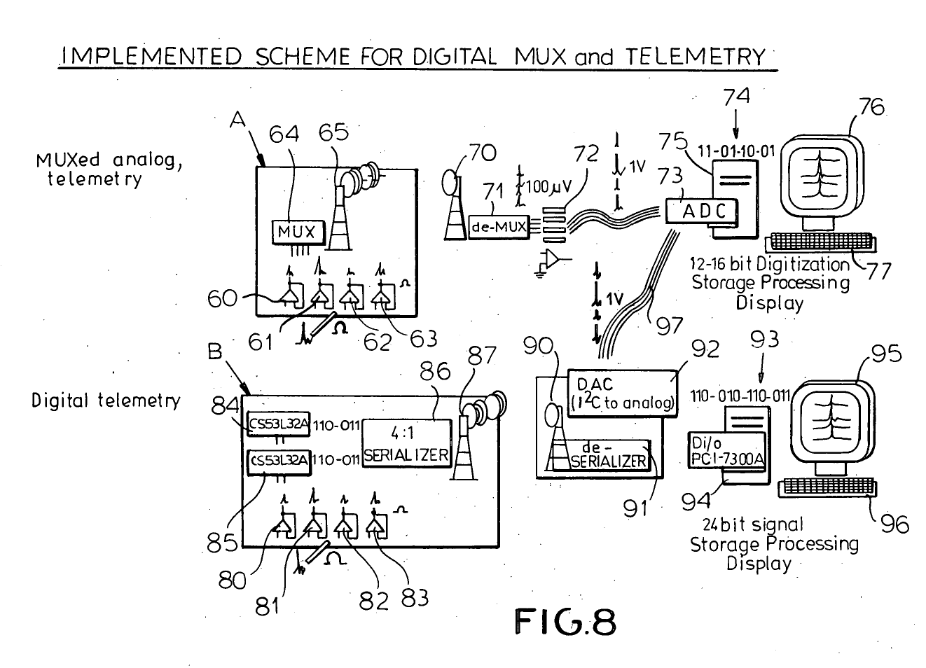

In FIG. 8, the overall system has been shown in greater detail. In the transmitter stage, four action potential outputs from conventional follower circuits are represented diagrammatically at 80, 81, 82 and 83. These signals represent any event transduced to an electrical signal. Each pair of signals is provided as analog inputs to two channel analog-to-digital converters 84, 85 etc. which are analogous to those of FIG. 2 providing inputs to the 4:1 serializer 86 which is equivalent to the multiplexer shown at 38 in FIG. 2 which feeds its output to the GHz transmitter 65 which transmits to a receiver 70. The serializer can be N:1, where N is a whole number, the maximum of which is limited by the frequency of the measurement (i.e. digitizing clock of the ADCs feeding into the serializer) and the clock speed of the audio standard by the equation ADC frequency×N=clock speed of the audio standard.

After error correction of the audio data stream by the receiver, the latter is connected to the deserializer 91 and, if desired for legacy analog signal processing, a digital to analog converter (DAC) 92. The deserializer can provide a direct digital output to the digital audio storage and processing unit 93 which can include a digital audio processor for hardware digital filling, a recorder 94, and display 95. When a digital-to-analog converter is included at the receiving station, the analog channels may be supplied at 97 to a conventional ADC 73 for legacy analog data acquisition 74 and processing represented by a computer/data recorder 75, monitor 76, and keyboard 77.

While an 8 channel system has been described, a 16 channel system can be used simply by duplicating the circuit of FIGS. 2 and 8 and feeding the 8 stereo ADC outputs to an 8:1 serializer. A system consisting of multiples of 16 channels can be realized by simply multiplying the 16-channel system. The limiting number of channels is determined ultimately by the clocking speed of the standard audio signal that the serializer is designed to conform to. In the case of the current 96 ksps I2S audio standard, 16, 6 kHz ADC outputs can be serialized onto one 96 kHz output. As the audio standards increase in clock speed for example to 192 ksps, then more channels, for example 32, ADC outputs can be serialized onto a single 192 ksps I2S output to realize a 32-channel module capable of recording action potentials with 6 kHz+band-width. If a slower signal is to be recorded then more channels sampled at lower frequency can be transmitted on the limiting throughput of the audio standard being utilized.

Because of the use of standard audio electronics and conformance of the serialized signals to digital audio standard format (e.g. I2S), the system described is of simple construction and circuitry and inexpensive cost and can be used for bioelectric signal telemetering for untethered live animal and human subjects without significant restriction of mobility.

We claim:

1. A method of monitoring neuronal activity comprising the steps of:
(a) connecting electrodes to neural activity points of an animal or human subject and obtaining analog signals representing activity at said points;
(b) securing to said animal subject a digital recording apparatus having mono or stereo audio analog-to-digital converters capable of digitizing and multiplexing two analog inputs each into respective single digital data streams which can be multiplexed by a serializing stage, and a gigahertz radiofrequency transmitter for said data streams and connecting said audio analog-to-digital converters to said electrodes, whereby digital data streams representing digitized and multiplexed signals from said electrodes are transmitted by said apparatus;
(c) at a receiver separated from said live subject receiving the digital data streams transmitted by said apparatus, demultiplexing channels of the received digital data streams to produce digital channels corresponding to the analog inputs, for digital data processing and/or converting said digital channels to analog outputs; and
(d) recording said digital and/or analog outputs in a multichannel recorders having recording channels assigned to the respective electrodes.

2. The method defined in claim 1, further comprising the step of serializing (digital multiplexing) the single digital data streams supplied to said gigahertz radio frequency transmitter.

3. The method defined in claim 2, further comprising the step of processing the received digital data stream of step (c) to monitor the activity at said points.

4. An apparatus for monitoring neuronal activity comprising:
a multiplicity of electrodes connected to neural activity points of a subject and obtaining analog signals representing activity at said points;
a digital recording apparatus mounted on said subject and having audio analog-to-digital converters connected to said electrodes and each capable of digitizing and multiplexing analog inputs each into respective single digital data streams, and a gigahertz radiofrequency transmitter for said data streams and connected to said audio analog-to-digital converters, whereby digital data streams representing digitized and multiplexed signals from said electrodes are transmitted by said apparatus;
a receiver separated from said animal subject receiving the digital data streams transmitted by said apparatus, and provided with a demultiplexer for demultiplexing channels of the received digital data streams to produce digital channels corresponding to the analog inputs, and digital-to-analog converters for converting said digital channels to analog outputs; and
a multichannel analog recorder connected to said receiver and having recording channels assigned to the respective electrodes.

5. The apparatus defined in claim 4 wherein each of said electrodes is connected to an operational and instrumentation amplifier common-mode noise-reduction stage.

6. The apparatus defined in claim 5, further comprising at least one further multiplexer for multiplexing a plurality of said signal digital data streams prior to transmission by said apparatus.

7. The apparatus defined in claim 6, further comprising a digital audio processor receiving said digital data streams for monitoring the neural activity at said points.

8. A method of monitoring bioelectrical activity in a living animal or human subject comprising the steps of:
(a) connecting electrodes to points of a living subject having bioelectrical activity and obtaining analog electrical signals representing said activity at said points;
(b) multiplexing electrical signals obtained at said points and transmitting a data stream represented by the multiplexed signals through a radio transmitter circuit to a radio receiver;
(c) demultiplexing a received data stream at said receiver; and
(d) monitoring the activity at said points based upon the demultiplexed data stream.
